(19) United States
(12) Patent Application Publication
(10) Pub. No.: US 2013/0172730 A1
(43) Pub. Date: Jul. 4, 2013

(54) Motion-Compensated Image Fusion

(71) Applicant: Amit Cohen, Binyamina (IL)

(72) Inventor: Amit Cohen, Binyamina (IL)

(21) Appl. No.: 13/339,588

(22) Filed: Dec. 29, 2011

(51) Int. Cl. A61B 6/00

(52) U.S. Cl. 600/424

(57) ABSTRACT

One aspect of the present disclosure involves a medical imaging system and a method for displaying images of medical devices deployed within a body of a patient in relation to images of anatomical structures of the body. Because of various operating constraints, the images may have been acquired from different coordinate systems at different times. Three-dimensional (3D) positions of physical and virtual sensors in addition to phases of a body organ may be used in associating the images from the different coordinate systems. The system may also compensate for any movement of the patient along an operating table, as well as for movement caused by respiratory and cardiac activity.

### BACKGROUND OF THE INVENTION

a. Field of the Invention

The present disclosure relates to medical imaging systems and methods for imaging medical devices and anatomies of patients. More particularly, the present disclosure relates to a medical imaging system and a method for displaying a medical device in relation to an anatomy of a patient's body.

b. Background Art

It is desirable for medical professionals to view an image of an anatomical structure of a patient when maneuvering interventional medical devices and performing therapy within the patient. Oftentimes, though, it is undesirable or even impossible to image the anatomy of the patient when maneuvering the medical devices within the patient. This is so because operating constraints associated with some body organs and blood vessels can prevent the simultaneous capture of images showing medical devices and images showing the anatomy, particularly where a contrast agent or special dye is utilized.

To illustrate, medical imaging systems may be used to assist with cardiac resynchronization therapy (CRT) implantation procedures. In such procedures, medical devices for delivering therapy and a left ventricular (LV) lead are typically advanced through a patient's coronary sinus ostium, where the ostium is the orifice of the coronary sinus. One way to obtain a representation of the coronary sinus is to take a venogram of the anatomy with a fluoroscopic imaging system. Contrast agent may be injected within the coronary sinus or other organ or blood vessels to facilitate the acquisition of the venogram with the imaging system. The contrast agent may even be trapped within the coronary sinus by positioning a balloon catheter within the coronary sinus ostium. The contrast agent highlights the anatomical structure of the coronary sinus on the venogram. Yet the balloon catheter must be removed before delivery tools such as guide wires and guide catheters, and the LV lead itself, are advanced through the coronary sinus ostium. Thereafter, the contrast agent may disperse from the coronary sinus. Thus, the beneficial effect of the contrast agent highlighting the anatomical structure can be lost when certain medical devices are navigated through the patient. This in turn means that the medical professional is prevented from acquiring images of the coronary sinus as certain medical devices are navigated through the patient.

Another example where it is difficult to image the anatomy when maneuvering medical devices within the patient comes from the field of coronary arterial interventions. There, medical professionals routinely use cine-loops with contrast agent to visualize the target coronary anatomy. But due to the adverse impact of contrast agents on the renal function of patients, diabetic patients in particular, medical professionals attempt to minimize the use of these substances. Thus, here too, medical professionals are unable to acquire images of the anatomy as certain medical devices are navigated through the patient.

One practice has been to use two displays, with one display showing “roadmaps” of the anatomy as previously imaged using the contrast agent trapped within the coronary sinus. The second display shows live images of the medical devices isolated from the anatomical structure. A medical professional, then, compares the two displays in an attempt to mentally associate the roadmaps of the anatomy with the live images of the medical devices.

This practice and others like it are of marginal benefit because they leave much unnecessary interpretation to the medical professional. Another drawback to this practice is that the imaged anatomy does not move with the real-time anatomical motion of the patient. Thus, juxtaposing live images of medical devices and previously-acquired anatomical images fails to account for patient movement along an operating table and localized tissue movement due to cardiac and respiratory activity.

Therefore, a system and a method are needed that enable viewing the medical devices in relation to the patient anatomy while compensating for movement of the anatomy between the time when the anatomy was imaged and the time when the medical devices perform therapy in the anatomy.

### SUMMARY OF THE INVENTION

The present disclosure involves a system and a method for displaying an image of a medical device deployed within a body of a patient in relation to an image of an anatomy of the body. Images of the anatomy may be acquired prior to their association with live images showing the medical device.

In some embodiments, the present disclosure includes an imager, a database, an anchor, a medical positioning system (MPS), a processor, and a display. The imager may utilize any imaging modality capable of capturing images of the anatomy of a body, images of a medical device within the body, or images of both the anatomy and a medical device within the body. On the other hand, multiple imagers utilizing different imaging modalities may also be used. That is, one imaging modality may be used to capture images in one time period, while another imaging modality may be used to capture images in another time period. The imager may acquire images of the anatomy of the body during a first time period. During a second time period, the imager may acquire additional images of the anatomy and/or images of a medical device, which may be inserted within the body at any point in time. Further, the system may store all images, and all information in general, in a database for future retrieval.

Different medical devices may serve different purposes, and some medical devices may serve more than one purpose. For example, some medical devices may be used purely as anchors for associating images from different coordinate systems. These medical devices may be maintained within the body throughout the first and second time periods. Other medical devices may be used to temporarily trap contrast agent within a particular body organ during the first time period. Still other medical devices may be used to locate or “place” virtual anchors at anatomical landmarks within the body during both time periods. Some medical devices may be used to deliver therapy within the body during the second time period.

In addition, the at least one anchor may operate with the MPS. The present disclosure contemplates using physical anchors, virtual anchors, and a combination of both physical and virtual anchors. Because the imager may acquire the images at different times and from different coordinate systems, while the anchors may serve multiple purposes, one of the primary purposes of the anchors as used in this invention is to serve as a common 3D position and orientation between numerous coordinate systems. This common 3D position and orientation, which may determined by the MPS, allows for the association or co-registration of numerous coordinate systems. If the anchor is a physical sensor affixed to a stable location along or within the body of the patient, the MPS may determine the position and orientation of the sensor when each image is acquired during the first and second time periods. If the anchor is virtual, the MPS may determine the position and orientation of the virtual anchor when a medical device with a physical sensor is positioned near an anatomical landmark in the first and second time periods.

The processor may associate the 3D positions and orientations of virtual and physical anchors with each image that is acquired during the first and second time periods. Because the anchors generally maintain the same positions and orientations in relation to the patient body in the first and second time periods, the processor may associate the first and second images using the 3D position and orientation of the anchors. Association of images to form the resultant image may involve making at least one of the images at least partially transparent and superimposing at least two images with one another.

Before presenting the resultant image, another aspect of the present disclosure involves motion compensating the images to account for cardiac and respiratory activity in addition to patient table motion that occurs between the times when the associated images are acquired. The processor may in some embodiments account for motion due to respiratory activity and patient table motion by analyzing the 3D positions and orientations of the physical and virtual anchors on or within the body at the times when the associated images are acquired. To account for cardiac activity between the times when the associated images are acquired, the system may employ organ sensors. Organ sensors may sense the phase of a patient's heart, for example, at the time when each image is acquired. Acquiring a set of images during the first time period may help ensure that the database contains images corresponding to a variety of cardiac phases. When an image is acquired in the second time period, therefore, the processor may select an image from the database that was acquired during the first time period so as to match the cardiac phase of the associated images.

Another aspect of the invention involves arranging the imager in similar positions and orientations during the first and second time periods. It can be helpful to match images from the first and second time periods that were acquired from substantially the same position and orientation with respect to the body. Position and orientation in this context may be measured with regard to the at least one anchor affixed to or within the patient body. In some embodiments, the processor may refrain from associating images where the position and orientation of the imager in the second time period do not sufficiently match a position and orientation in which the imager acquired images in the first time period.

It will be appreciated that in addition to the structure of the system, another exemplary aspect of the present disclosure is a method for displaying images of the anatomy of the body in relation to the position of a medical device and/or in relation to other images of the anatomy. It will be further appreciated that the methodology and constituent steps thereof, as described in some detail above, apply to this aspect of the disclosure with equal force. The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1A is a functional block diagram of a system for associating a first image acquired by a first imager with a second image acquired by a second imager.

FIG. 1B is a functional block diagram of each of the first medical positioning system (MPS) and the second MPS of the system of FIG. 1A.

FIG. 1C is a schematic illustration of a portion of the system of FIG. 1A that acquires the first image of a body of a patient.

FIG. 1D is a schematic illustration of another portion of the system of FIG. 1A that acquires the second image of the body of the patient and associates the first image with the second image.

FIG. 2 is a schematic illustration of an exemplary system for acquiring an image of the body of the patient and any images of medical devices disposed within the body.

FIG. 3 is a diagrammatic view of a medical device equipped with a sensor used, at least in part, to identify an anatomical landmark within or near a region of interest.

FIG. 4 is a diagrammatic view of anatomical landmarks superimposed on a two-dimensional (2D) image.

FIG. 5A is a schematic illustration of two reference sensors arranged on the body of a patient, the sensors helping to determine the scale factor of an image.

FIG. 5B is a schematic illustration of a first image of the body of the patient, as acquired by a first imager similar to the first imager of FIG. 1A.

FIG. 5C is a schematic illustration of a second image of the body of the patient, as acquired by a second imager similar to the second imager of FIG. 1A, wherein the scale of the second image is different from the scale of the first image of FIG. 5B.

FIG. 5D is a schematic illustration of the first image of FIG. 5B, corrected according to the scale of the second image of FIG. 5C.

FIG. 6A is a functional block diagram of a system for associating a first image acquired by a first imager with a second image acquired by a second imager.

FIG. 6B is a schematic illustration of a portion of the system of FIG. 6A that acquires the first image and detects a signal from an organ of the body of the patient.

FIG. 6C is a schematic illustration of another portion of the system of FIG. 6A that acquires the second image, detects a signal from an organ of the body of the patient, and associates the second image with the first image.

FIG. 7 is a flow diagram representing one possible method of operating the system for displaying images of a medical device in relation to images of anatomical structures.

FIG. 8A is a schematic illustration of a coronary sinus anatomy of the body of the patient, two medical devices disposed within the body, and several anatomical landmarks superimposed on the image.

FIG. 8B is a schematic illustration of two medical devices disposed within the body of the patient in addition to several anatomical landmarks superimposed on the image.

FIG. 8C is a resultant image based on the images shown in FIGS. 8A-8B, with at least one of the images being superimposed with the other image so as to show the medical devices in relation to the coronary sinus anatomy.

### DETAILED DESCRIPTION OF THE INVENTION

In the following description, wherein like reference numerals are used to identify like components in the various views, a coordinate system can be orthogonal, polar, cylindrical, and so on. The term “image” refers to any type of visual representation of a portion of a body of a patient, either acquired directly or reconstructed from raw measurements. Such an image can be provided in one, two, or three spatial dimensions; a still image; or developing in time. Any medical positioning system (MPS) mentioned herein may be coupled with other devices or systems associated therewith, either physically (i.e., in a fixed location with respect thereto) or logically (i.e., where both collaborate within the same coordinate system). In the following description, a medical device can be a catheter (e.g., balloon catheter, stent catheter, surgical catheter, dilution catheter), a drug delivery unit (e.g., needle, catheter having a coated stent or a balloon, brachytherapy unit), a tissue severing unit (e.g., forceps, ablation catheter), and the like.

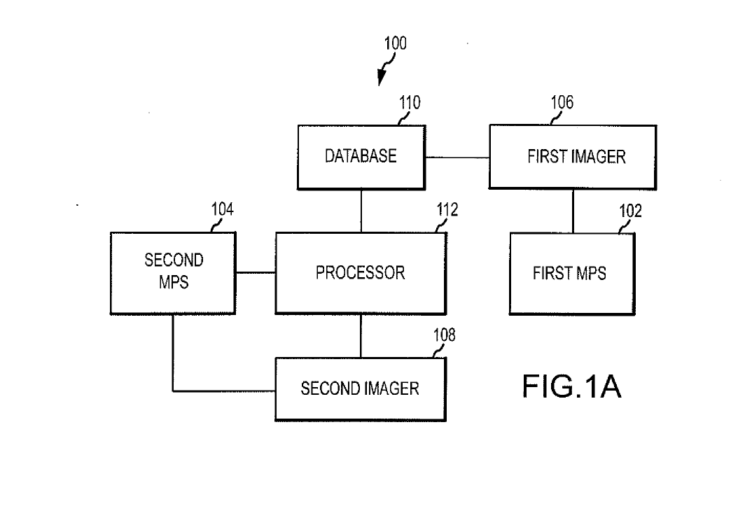

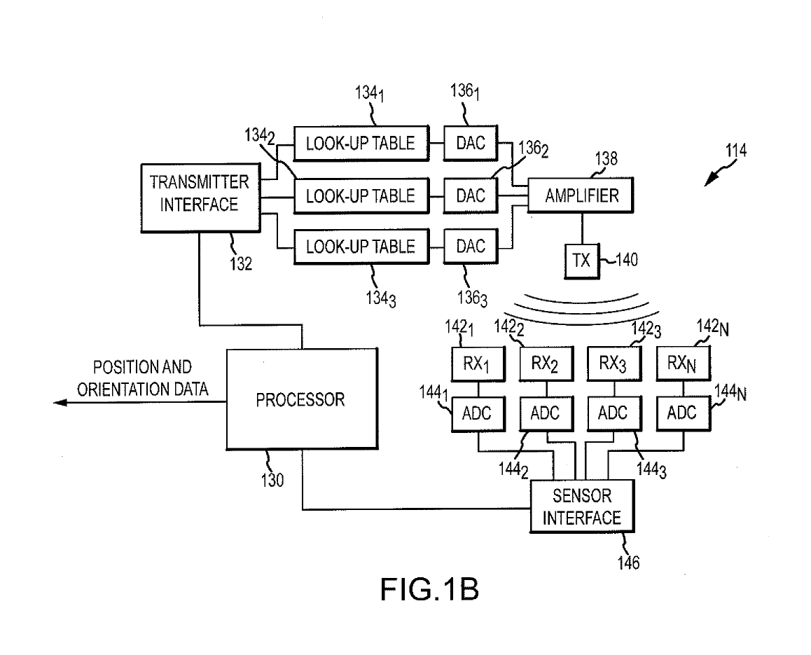

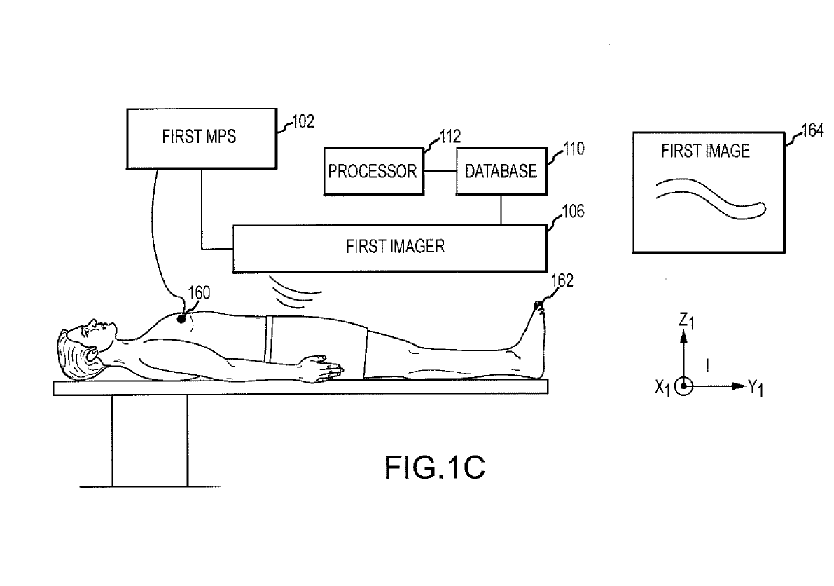

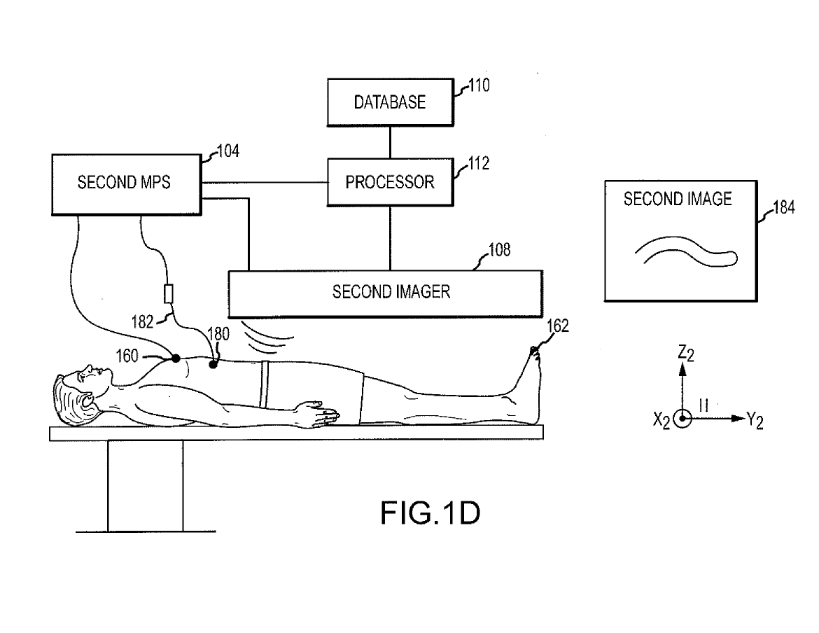

FIG. 1A is a functional block diagram of a system, generally referenced 100, for associating a first image acquired by a first imager with a second image acquired by a second imager. FIG. 1B is a functional block diagram of an exemplary MPS of the system 100 of FIG. 1A, and FIG. 1C is a schematic illustration of a portion of the system 100 that acquires the first image. FIG. 1D is a schematic illustration of another portion of the system 100 that acquires the second image and associates the first image with the second image.

With reference to FIG. 1A, the system 100 may generally include a first MPS 102, a second MPS 104, a first imager 106, a second imager 108, a database 110, and a processor 112. Each of the first MPS 102 and the second MPS 104, generally referenced 114 (shown in FIG. 1B), may be a device that determines, among other things, the position and orientation (P/O) of at least one sensor. The MPS 114 may be similar to the MPS disclosed in U.S. Pat. No. 6,233,476 to Strommer et al., entitled “MEDICAL POSITIONING SYSTEM,” which is hereby incorporated by reference in its entirety. In general, the first MPS 102 is used during a first time period, and the second MPS 104 is used during a second time period. In some embodiments, however, the first MPS 102 may be the same apparatus as the second MPS 104, though operated at different times—and referred to separately for clarity. In other embodiments, the first MPS 102 is in fact a different apparatus than the second MPS 104. Similarly, the first imager 106 may in some embodiments be the same apparatus as the second imager 108, though operated at different times—and referred to separately for clarity. In other embodiments, the first imager 106 is in fact a different apparatus than the second imager 108. Further, it should be noted that FIG. 1A, like all other figures, is merely exemplary and non-limiting. For example, another embodiment of the system 100 may include a direct connection between the first MPS 102 and the processor 112 and a direct connection between the second imager 108 and the database 110.

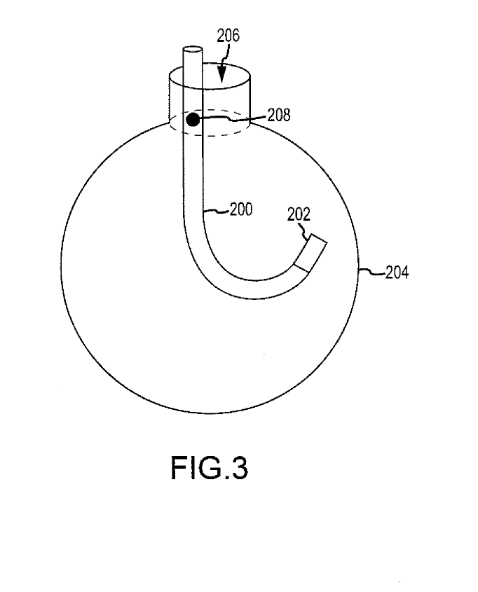

As stated above, the MPS 114 may determine the P/Os of one or more sensors. Each P/O determination may include at least one of a position and an orientation relative to a reference coordinate system, which may be the coordinate system of the MPS 114. P/O can be tracked to the relevant number of degrees of freedom according to the application and imaging. For example, the P/O may be expressed as a position (i.e., a coordinate in three axes X, Y, and Z) and an orientation (i.e., an azimuth, elevation, and potentially roll) of a magnetic field sensor in a magnetic field relative to a magnetic field generator(s) or transmitter(s). Other expressions of P/O (e.g., other coordinate systems such as position [X, Y, Z] and orientation angles [α, β, χ]) are known in the art and fall within the spirit and scope of the present disclosure (see, e.g., FIG. 3 and the associated text of U.S. Pat. No. 7,343,195 to Strommer et al., entitled “METHOD AND APPARATUS FOR REAL TIME QUANTITATIVE THREE-DIMENSIONAL IMAGE RECONSTRUCTION OF A MOVING ORGAN AND INTRA-BODY NAVIGATION,” which is hereby incorporated by reference in its entirety). Other representations of P/O may be used, with respect to other coordinate systems in use.

One way to determine the three-dimensional (3D) P/Os of the sensors in the reference coordinate system is for the MPS 114 to capture and process signals received from these sensors while such sensors are disposed, for example, in a controlled low-strength AC magnetic field. Each sensor may comprise one or more magnetic field detection coil(s), and variations as to the number of coils, their geometries, spatial relationships, the existence or absence of cores, and the like are possible. From an electromagnetic perspective, the sensors may develop a voltage that is induced on the coil residing in a changing magnetic field, as contemplated here. The sensors may be configured to detect one or more characteristics of the field(s) in which they are disposed and to generate an indicative signal, which may be further processed by the MPS 114 to obtain a respective P/O thereof. One such exemplary sensor is disclosed in U.S. Pat. No. 7,197,354 to Sobe, entitled “SYSTEM FOR DETERMINING THE P/O OF A CATHETER,” which is hereby incorporated by reference in its entirety. Even though the present disclosure mentions the use of one or more magnetic-based MPSs and sensors, the present disclosure contemplates using one or more MPSs and sensors that operate with other modalities as well. Likewise, even though multiple sensors are used in some figures and examples, associating images based on the 3D P/Os of a single sensor is within the scope of the present disclosure.

Further, the database 110 may be a data storage unit that allows for storage and access of data records. The database 110 may be, for example, a magnetic memory unit (e.g., floppy diskette, hard disk, magnetic tape), optical memory unit (e.g., compact disk), volatile electronic memory unit (e.g., random access memory), non-volatile electronic memory unit (e.g., read only memory, flash memory), remote network storage unit, and the like. The database 110 may store data required by the system 100 such as, for example and without limitation, frames of captured two-dimensional (2D) images from the first and second imagers 106, 108 as well as MPS sensor readings from the MPS 114. Data may be transferred to the database 110, from which the data may be recalled for processing. Intermediate and final data values obtained throughout computations of a processor may also be stored in the database 110. The database 110 may store further information from additional devices used in combination with the system 100 (e.g., information from an external monitoring device such as an electro-cardiogram (ECG) monitor, intravascular ultrasound information, and the like). In general, the database 110 may store all possible information that is needed by the system 100.

With respect to the first and second imagers 106, 108, each imager may be a device that acquires an image of the body of a patient (not shown). The first imager 106 may be coupled with the first MPS 102 and with the database 110, while the second imager 108 may be coupled with the second MPS 104 and, in some embodiments, with the database 110. The first and second imagers 106, 108 can include any type of image acquisition system known in the art, such as, for example and without limitation, ultrasound, inner-vascular ultrasound, X-ray, C-Arm machines (equipped with such devices), fluoroscopy, angiography, computerized tomography (CT), nuclear magnetic resonance (NMR), positron-emission tomography, single-photon emission tomography, optical imaging, nuclear imaging—PET, thermography, and the like. Notably, the first imager 106 and the second imager 108 may or may not be the same type of imaging system or use the same imaging modality. For example, the first imager 106 may be a fluoroscopic X-ray device, and the second imager 108 may be a traditional X-ray device. These exemplary image acquisition systems, moreover, may acquire images with respect to an image coordinate system.

The positional relationship between the image coordinate system and the reference coordinate system may be calculated based on a known optical-magnetic calibration of the system (e.g., established during setup). This calibration is possible because the positioning system and the imaging system may be fixed relative to one other in some embodiments. That is, the first imager 106 may be fixed relative to the first MPS 102, and the second imager 108 may be fixed relative to the second MPS 104. One way for the MPS 114 to determine the P/Os of the imagers is by affixing sensors to (i.e., to, within, about, etc.) the imagers. By determining the positional relationship between the first imager 106 and the first MPS 102, the processor 112 may co-register or otherwise associate the images of the first imager 106 and the sensors measured by the first MPS 102 within a common coordinate system. The same may be true for the images of the second imager 108 and the sensors measured by the second MPS 104.

Although the present disclosure hereinafter refers to the reference coordinate system (as opposed to the imaging coordinate system) as if it were the default coordinate system, these references are merely for the sake of clarity and consistency. Because of the interchangeability of the reference and imaging coordinate systems, this disclosure could likewise refer to the imaging coordinate system as if it were the default coordinate system.

Associating images and sensor readings from the first MPS 102 and the first imager 106 with images and sensor readings from the second MPS 104 and the second imager 108 can also be advantageous. The processor 112, which may, for example, be a central processing unit (CPU), is one apparatus that can perform this process and associate such data, as described more fully below. As shown in FIG. 1A, the processor 112 may in one embodiment be coupled with the database 110, with the second imager 108, and with the second MPS 104. The processor 112 may be similar to the main computer of U.S. Pat. No. 7,386,339 to Strommer, entitled “MEDICAL IMAGING AND NAVIGATION SYSTEM,” which is herein incorporated by reference. The processor 112 may co-register, superimpose, fuse, or otherwise associate data and images from the first imager 106 and the first MPS 102 with data and images from the second imager 108 and the second MPS 104. In addition, the processor 112 may perform necessary calculations; correlate between the different data streams; perform filtering, segmentation, and reconstruction of 2D and 3D models; and conduct other operations.

Once the numerous coordinate systems are registered, a P/O in one coordinate system may be transformed into a corresponding P/O in another coordinate system through the transformations established during the registration process, a process known generally in the art, for example as described in U.S. Patent Publication No. 2006/0058647 to Strommer et al., entitled “METHOD AND SYSTEM FOR DELIVERING A MEDICAL DEVICE TO A SELECTED POSITION WITHIN A LUMEN,” hereby incorporated by reference in its entirety.

In some embodiments, the procedures performed during the first time period may take place at a location different than that where the procedures of the second time period take place. In these embodiments, the first imager 106 and the first MPS 102 may be different equipment, respectively, than the second imager 108 and the second MPS 104. If so, the data acquired during the first and second time periods may be associated via transmission over a network (e.g., LAN, WAN, wired or wireless).

The system 100 may further include a display (not shown) for presenting resultant images, motion pictures, or image sequences of the inspected organ in real-time, for example. A motion picture may consist of a series of 2D images captured by the first and second imagers 106, 108. Where a medical device inserted within the body of the patient is radio-opaque, the motion picture may also display the shape of the medical device as it is guided within the patient body, respective of different activity-states of an inspected organ, as described below. The display may further present a selected image frame of the motion picture respective of the real-time detected organ activity-state. In addition, the display may provide different playback effects, freeze frames, change speed, select features, etc.

For example, the display may present a playback of previous images in a sequence, showing the progress of the medical device during previous activity states of the organ. The display may include multiple monitors, or separate windows within a single monitor, where each monitor or window presents a different view. As a further example, one monitor or window may present the current position of the medical device in the current image frame of the inspected organ respective of the current activity-state, while another monitor or window may present the current position of the medical device in a previous image frame (or image sequence) of the inspected organ respective of a previous activity-state (or activity-states). The display may be a 2D display, an auto-stereoscopic display to be viewed with a suitable pair of spectacles, a stand alone stereoscopic display, a pair of goggles, and the like. Still further, the display may present resultant images showing the current shape and position of a medical device in a current image frame in relation to the anatomy of a patient recorded in a previous image frame.

To associate data from the first imager 106 and the first MPS 102 collected during the first time period with data from the second imager 108 and the second MPS 104 collected during the second time period, the system 100 may use 3D P/Os of physical and virtual anchors. The anchors serve as common P/Os by which the processor 112 associates data from the first and second time periods.

One merely exemplary way of determining the 3D P/Os of sensors, some of which may serve as physical anchors, is by way of the configuration shown in FIG. 1B. As shown, the MPS 114 may include a processor 130; a transmitter interface 132; a plurality of look-up table units 134₁, 134₂, and 134₃; a plurality of digital to analog converters (DACs) 136₁, 136₂, and 136₃; an amplifier 138; a transmitter 140 (TX); a plurality of sensors 142₁ (RX₁), 142₂ (RX₂), 142₃ (RX₃), and 142_{N} (RX_{N}); a plurality of analog to digital converters (ADCs) 144₁, 144₂, 144₃, and 144_{N}; and a sensor interface 146. The processor 130 of FIG. 1B is shown and described as being separate and distinct from the processor 112 shown in FIG. 1A. In some embodiments, though, the MPS 114 of FIG. 1B may share the processor 112 shown in FIG. 1A, as opposed to having its own processor 130.

The transmitter interface 132 may be coupled with the processor 130 and with the look-up table units 134₁, 134₂, and 134₃. The DAC units 136₁, 136₂, and 136₃ may be coupled with a respective one of look-up table units 134₁, 134₂, and 134₃ and with the amplifier 138. The amplifier 138 may further be coupled with the transmitter 140. Also, the sensors described throughout the present disclosure may be like the sensors 142₁, 142₂, 142₃, and 142_{N}, regardless of whether the sensors are referred to or referenced differently.

The ADCs 144₁, 144₂, 144₃, and 144_{N} may be respectively coupled with the sensors 142₁, 142₂, 142₃, and 142_{N} and with the sensor interface 146. The sensor interface 146 may be further coupled with the processor 130.

Each of the look-up table units 134₁, 134₂, and 134₃ may produce a cyclic sequence of numbers and may provide them to the respective DAC unit 136₁, 136₂, and 136₃, which in turn translates them to a respective analog signal. Each of the analog signals may be respective of a different spatial axis. In the present example, the look-up table 134₁ and the DAC unit 136₁ may produce a signal for the X axis, the look-up table 134₂ and the DAC unit 136₂ may produce a signal for the Y axis, and the look-up table 134₃ and the DAC unit 136₃ may produce a signal for the Z axis.

The DAC units 136₁, 136₂, and 136₃ may provide their respective analog signals to the amplifier 138, which amplifies and provides the amplified signals to the transmitter 140. The transmitter 140 may provide a multiple axis electromagnetic field, which can be detected by the sensors 142₁, 142₂, 142₃, and 142_{N}. Each of the sensors 142₁, 142₂, 142₃, and 142_{N} may detect an electromagnetic field, produce a respective electrical analog signal, and provide the respective electrical analog signal to the respective ADC unit 144₁, 144₂, 144₃, and 144_{N} coupled therewith. Each of the ADC units 144₁, 144₂, 144₃, and 144_{N} may digitize the analog signal fed thereto, convert the analog signal to a sequence of numbers, and provide the sequence of numbers to the sensor interface 146, which may in turn provide the sequence of numbers to the processor 130. The processor 130 may analyze the received sequences of numbers and thereby determine the P/O of each of the sensors 142₁, 142₂, 142₃, and 142_{N}. The processor 130 may further determine distortion events and update the look-up tables 134₁, 134₂, and 134₃, accordingly.

With reference to FIG. 1C, the first MPS 102 may be coupled with a patient reference sensor (PRS) 160 and with the first imager 106, which may in turn be coupled with the database 110. The PRS 160, which may be similar to one of the sensors 142₁, 142₂, 142₃, and 142_{N} of FIG. 1B, may be attached to the body of a patient 162, and the PRS 160 may also be similar to one of the sensors disclosed in U.S. Pat. No. 6,233,476 to Strommer et al., entitled “MEDICAL POSITIONING SYSTEM,” incorporated by reference above. The PRS 160 may be attached to the skin (not shown), placed under the skin, or implanted within the body of the patient 162. Thus, the PRS 160 may be affixed to the body of the patient 162 and can maintain substantially the same P/O relative to the body of the patient 162 throughout the first and second time periods. One exemplary location for attachment of the PRS 160 is a patient's manubrium sternum, which is a stable place on the chest of the patient 162. Variations and combinations of the foregoing are also possible, for example, including the use of multiple PRSs and the use of a PRS in a location other than on the patient chest. The PRS 160 can be wired and may include a connector (not shown) in order to disconnect the PRS 160 from the first MPS 102 and connect the PRS 160 to the second MPS 104, that is, in embodiments where the first and second MPSs 102, 104 are different devices. Alternatively, the PRS 160 may operate wirelessly.

The first MPS 102 may be associated with an X₁, Y₁, Z₁ reference coordinate system (i.e., reference coordinate system I). The first imager 106 may be calibrated with the first MPS 102, as described above, such that the P/O of the first imager 106 may be defined relative to the reference coordinate system I. Based on an electromagnetic field generated by the first MPS 102, the PRS 160 may provide a signal representative of its P/O to the first MPS 102. The first MPS 102 may thereby determine the P/O of the PRS 160 in the reference coordinate system I. The first MPS 102 may provide signals representative of the P/Os of the PRS 160 and the first imager 106 to the first imager 106. At or around the same time, the first imager 106 may acquire a first image 164 of the body of the patient 162. The first imager 106 may store in the database 110 the first image 164, the P/O of the first imager 106 when the first image 164 was acquired, and the P/O of the PRS 160 when the first image 164 was acquired. The determined P/Os may be recorded to the database 110 with respect to reference coordinate system I.

The present disclosure contemplates variations of this arrangement as well. For example, the first MPS 102 may be coupled to the image database 110 and may directly supply the P/Os of the PRS 160 and the first imager 106 to the image database 110. The processor, then, could oversee this procedure, the timing of the procedure, and coordination of the various devices.

Moreover, during this first time period, the first imager 106 and the first MPS 102 may repeat this procedure and acquire a first set of images where the first imager 106 is arranged at a variety of P/Os in relation to the body of the patient 162. Still further, the first imager 106 and the first MPS 102 may acquire numerous images at each of the variety of P/Os. The acquisition of numerous images becomes helpful during the second time period, where images from the first time period are retrieved from the database 110 and associated with images from the second time period based upon contraction states of the heart, lungs, and other organs. Thus, the database 110 may store a variety of types of images including, for example, one or more 2D still images acquired at various times in the past or a plurality of related 2D images obtained in real-time from an imager wherein the database 110 acts as a buffer. Another group of images may include a sequence of related 2D images defining a cine-loop wherein each image in the sequence has at least an ECG timing parameter associated therewith adequate to allow playback of the sequence in accordance with acquired real-time ECG signals obtained from an ECG monitor.

With reference to FIG. 1D, the second MPS 104 is shown to be coupled with the PRS 160, with the second imager 108, with the processor 112, and with a medical device sensor 180 attached to at least one medical device 182 inserted within the body of the patient 162. The medical device 182 and the medical device sensor 180 are described below. As previously described, however, the processor 112 may also be coupled to the database 110 and to the second imager 108.

The second imager 108 may acquire a second image 184 or second set of images of the body of the patient 162, typically while a medical professional is performing a medical operation on the patient 162. The second MPS 104 may be associated with an X₂, Y₂, Z₂ reference coordinate system (i.e., reference coordinate system II). The second imager 108 may be calibrated with the second MPS 104 such that the P/O of the second imager 108 may be defined relative to reference coordinate system II. Similar to the technique of the first time period, the second MPS 104 may generate an electromagnetic field so that the PRS 160 may provide a signal representative of its P/O to the second MPS 104, with respect to the reference coordinate system II. The medical device sensor 180, sensors affixed to the second imager 108, and any other sensors may also provide signals representative of their P/Os to the second MPS 104. The P/Os of the sensors may then be sent from the MPS 114 to the processor 112 or to the database 110.

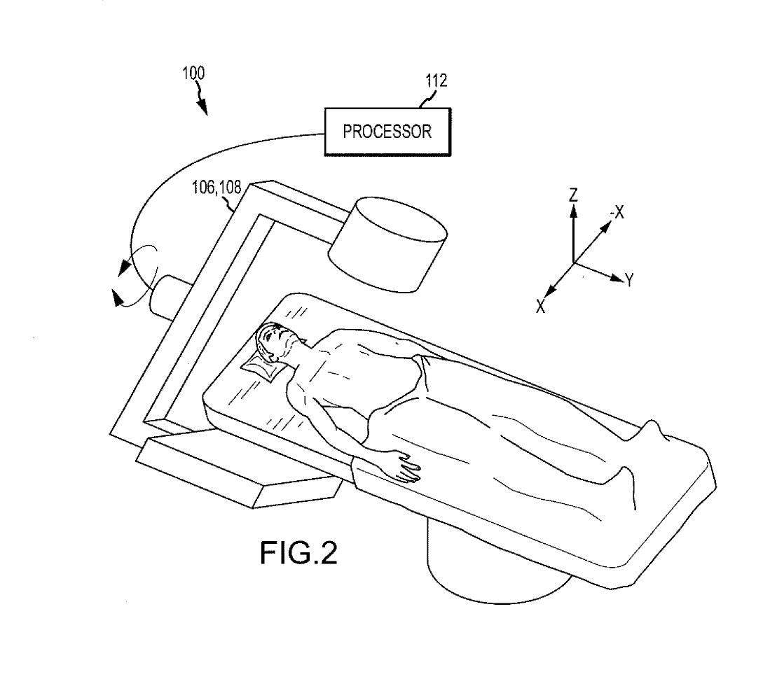

To facilitate the association of 2D images from the first and second time periods, it may be advantageous to arrange the second imager 108 in the P/Os in which the first imager 106 acquired images during the first time period. Therefore, the processor 112 may arrange the second imager 108 in P/Os that are substantially identical, or at least similar, to the P/Os in which the first imager 106 acquired images of the patient 162. “Matching” the P/Os of the first and second imagers 106, 108 may be easiest where the first and second imagers 106, 108 are either the same device or are maneuvered similarly, as shown in FIG. 2. Such maneuvering may be controlled by the processor 112. These matching P/Os could be relative to the P/O of the PRS 160 and/or other reference sensors on or in the body of the patient 162. In some embodiments, the system 100 will refrain from associating images from the first and second time periods until the P/O of the second imager 108 sufficiently corresponds to a P/O in which the first imager 106 acquired images.

With further reference to FIG. 1D, at least one medical device 182 may be inserted into the body of the patient 162 at any point in time, whether during or prior to the first or second time periods. Medical devices can be used in a number of different ways—beyond those mentioned above (e.g., as a balloon catheter, as a surgical catheter, as a needle, as a brachytherapy unit). For one, sensors (e.g., medical device sensor 180) on or along the medical devices may be used as anchors if maintained within the patient during both the first and second time periods. Second, the medical devices may be radio-opaque such that the medical devices are visible on images that are acquired. Third, sensors affixed to the medical devices may be used to “learn” the motions experienced in the body of the patient 162 due to respiratory and cardiac activity. Fourth, the medical devices may be used to place virtual anchors within the body of the patient 162, as described below.

Further, numerous medical devices may be used during the first or second time periods. Some medical devices may be inserted into the body of the patient for only one of the time periods, while other medical devices may remain in the patient 162 throughout the first and second time periods. Likewise, some medical devices may be used actively, while others are used passively. For example, in some contexts, a reference catheter may be considered to be generally passive, while an ablation catheter may be considered to be generally active.

Locating Anchors for Association of Data from Different Coordinate Systems.

As described above, associating data acquired during the first time period with data acquired from the second time period can be advantageous. The processor 112 is one apparatus that may associate data from the first and second time periods. Specifically, the processor 112 may transform (e.g., rotate and translate) and scale coordinates from reference coordinate system I to reference coordinate system II, or from reference coordinate system II to reference coordinate system I. The processor 112 may associate the first image 164 and the second image 184 by superimposing one image onto, over, behind, or within the other image and using transparency, translucency, and the like in at least one of the images to produce a resultant image. But to associate images acquired from two or more coordinate systems, the system 100 needs the 3D P/O of at least one anchor that is common to the different coordinate systems.

An anchor may be, for example and without limitation, any object, location, implant, anatomical feature, or a combination of the same that maintains the same P/O with respect to the patient body between the first and second time periods. By way of the anchor, the system may compute at least one transformation matrix to transform data from one coordinate system to another. Two types of exemplary anchors include physical anchors and virtual anchors. The present disclosure contemplates using physical anchors, virtual anchors, or a combination of physical and virtual anchors. Further, more anchors may provide a more-robust association of data from multiple coordinate systems.

Any one or more of the aforementioned sensors may serve as a physical anchor. With reference to FIG. 1C, for example, the PRS 160 could serve as an anchor for registration of multiple coordinate systems since it may be attached to a patient's manubrium sternum, which is a stable place on the chest that would remain substantially fixed between the first time period and the second time period. Another example of a physical anchor is where the medical device 182 serves as a reference catheter, and the medical device sensor 180 is located at a distal end of the reference catheter. The medical device 182 may be positioned within the patient 162 before images are acquired during the first time period. If the medical device 182 is maintained in place through the second time period, the MPS 114 may determine the P/O of the medical device sensor 180 for use as an anchor point between reference coordinate system I and reference coordinate system II. The medical device sensor 180 could be used in addition or in the alternative to the PRS 160 serving as an anchor.

In the alternative or in addition to physical anchors, virtual anchors may also be used to facilitate the association of data from one coordinate system to another. Virtual anchors may be located or “placed” at anatomical landmarks that are identifiable to a user of the system 100 and remain fixed or substantially fixed with respect to the patient body between the first and second time periods. The virtual anchors may be similar to those discussed in U.S. Patent Publication No. 2011/0054308 to Cohen et al., entitled “METHOD AND SYSTEM FOR SUPERIMPOSING VIRTUAL ANATOMICAL LANDMARKS ON AN IMAGE,” which is hereby incorporated by reference in its entirety.

In the example shown in FIG. 3, a medical device takes the form of a catheter 200 having a medical device sensor 202 at a distal end. The catheter 200 may be maneuvered by a medical professional towards a desired region of interest 204 (e.g., the right atrium of the heart) contained within the patient's body. Maneuvering to the region of interest 204 may involve passing the catheter 200 through an insertion region 206 (i.e., in this example where the destination site is the right atrium, the Superior Vena Cava (SVC) is the insertion region). Here, the SVC 206 may constitute the anatomical landmark where the virtual anchor is located. The user may visually detect when the catheter tip is positioned near the anatomical landmark (SVC 206). For example, a radio-opaque medical device inserted within the patient's body is visible on images of the body. When the medical professional believes that the catheter is at the desired landmark, he or she marks a location 208 of the catheter tip, as determined in accordance with the output of the medical device sensor 202, and thus also the location of the anatomical landmark, through interaction with a user interface. To supplement recognition, the system 100 may be optionally configured in an embodiment to superimpose a representation of the catheter's tip location on the image being displayed to the user, for example, in the form of cross-hairs or the like.

The system 100 may be configured generally to present a user interface configured to allow a user (e.g., a medical professional) to designate when the medical device has been maneuvered to a desired point in the region of interest where the virtual anchor is to be established, as described with reference to FIG. 3. The user interface may operate with the display. The system 100 may be further configured to record the P/O of the medical device sensors of the medical device when so indicated by the user. The user may interface with the system 100 through input/output mechanisms including, for example, a keyboard, a mouse, a tablet, a foot pedal, a switch, or the like. More specifically, the user interface may be a graphical user interface (GUI), for example, configured to receive the user's “mark” as an input signal constituting the request to locate the anchor and record the P/O reading. The signal may take the form of some user-initiated action such as actuation of a joystick, a push button, a pointing device (e.g., mouse, stylus and digital tablet, track-ball, touch pad), or by any other means. In this example, the user interface may recognize the user request, and the system 100 may then record the P/O reading corresponding to the location 208.

To facilitate marking the desired anchor in the region of interest, the system 100 may be configured to perform the following general steps: (i) presenting the image of the region of interest on the display; (ii) receiving an indication from the user when a sensor of a radio-opaque medical device is positioned at an anatomical landmark within the region of interest; and (iii) determining and recording the 3D P/O of the sensor in the reference coordinate system of the MPS 114.

By identifying in the first and second time periods the anatomical location that serves as the virtual anchor, the system may associate data from the coordinate systems of the first and second time periods. Moreover, the processor may be configured to modify the recorded P/O reading of a virtual anchor, per the techniques disclosed below or via other motion-compensation techniques. Modification of the P/O reading may be desirable so as to account for patient body, respiration, and cardiac-related motions between the first time period when the anatomical landmark is first located and the second time period when the anatomical landmark is again located.

In an embodiment, the system 100 may be configured to allow a user to adjust the virtual anchor (e.g., to correct the anatomical landmark, if needed or desired). Further, the system 100 may also be configured to allow manual manipulation of coordinate system registration once data from multiple coordinate systems have been associated. For example, if the user recognizes that the resultant image is misaligned by two centimeters, the user may control the user interface to correct the misalignment.

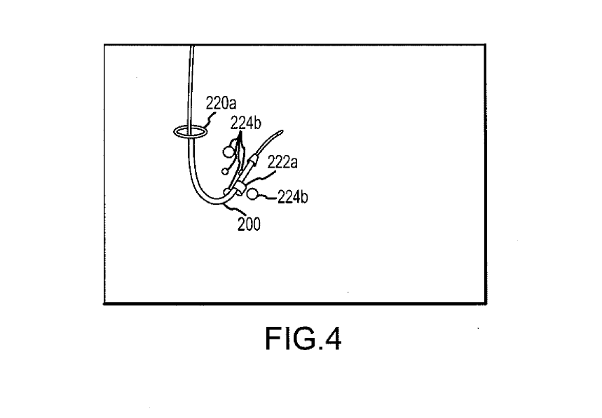

Virtual anchors may be superimposed on an image of the body if a user prefers. In some cases a graphic representation of the virtual anchor corresponds to a feature of the anatomy. For example, as shown in FIG. 4, an SVC landmark 220a may be a torus about the diameter of the actual SVC, while a virtual landmark 222a for the coronary sinus ostium may take the shape of a short cylinder about the diameter of a coronary sinus ostium. Additional anchors 224b (represented by spheres) are shown that do not necessarily relate to any specific anatomical location, shape, and/or size. Once the anatomical landmarks are identified as anchors in the first time period, the user may then later identify these anatomical locations in the second time period. Thereafter, the system 100 may perform motion compensation and use these virtual anchors to associate data from reference coordinate system I with data from reference coordinate system II. As a result, two or more images of the anatomy may be associated, in addition to a representation or image showing one or more medical devices. The resultant displayed images are thereby enhanced with more definition and with a view of the medical device in relation to the patient anatomy.

Scaling of the First and Second Images.

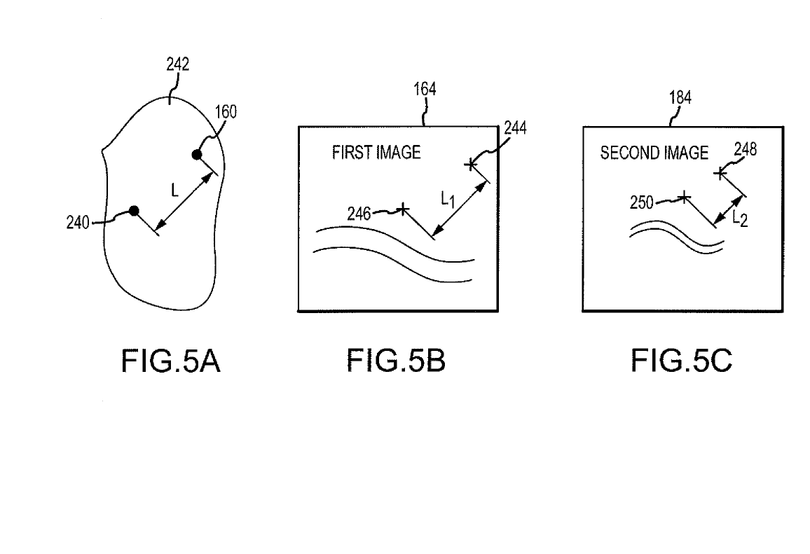

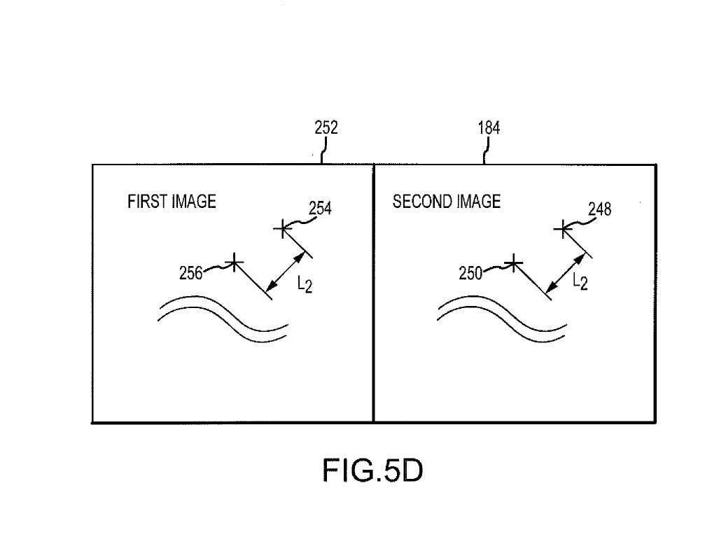

Although the second imager is preferably arranged at the same P/Os in which the first imager acquired images of the body of the patient, it is still possible that the scale of reference coordinate system I is different than that of reference coordinate system II. Therefore, the processor can change the scale of the first image according to the scale factor between reference coordinate system I and reference coordinate system II. The scale factor may be stored in processor or in the database. The system may use the PRS and one other anchor point to determine the scale factor between reference coordinate systems I and II. In the alternative, more than one PRS may be employed, as described herein below in connection with FIGS. 5A, 5B, 5C, and 5D.

FIG. 5A is a schematic illustration of two PRSs arranged on the body of a patient. The PRSs may be used to determine the scale factor of an image, according to a further embodiment of the disclosed technique. FIG. 5B is a schematic illustration of a first image of the body of the patient, acquired by a first imager, which may be similar to the first imager of FIG. 1A. FIG. 5C is a schematic illustration of a second image of the body of the patient, acquired by a second imager, which may be similar to the second imager of FIG. 1A. The scale of the second image in FIG. 5C may be different from the scale of the first image in FIG. 5B. FIG. 5D is a schematic illustration of the first image of FIG. 5B, corrected according to the scale of the second image of FIG. 5C.

As shown in FIG. 5A, the PRS 160 and another PRS 240 may be attached to a body 242 of a patient. The distance between the PRSs 160, 240 may be designated by the letter L. Further, each of the PRSs 160, 240 may be attached to the body 242 in a way similar to the way PRS 160 is attached to the body of patient 162. The PRSs 160, 240 may be incorporated with a system, such as system 100. Hence, the PRSs 160, 240 may be coupled with a first MPS during the first time period and with a second MPS during the second time period and/or while a medical operation is performed on the patient. Yet a registering module, with which a second imager may be coupled, may not be aware of the scale factor between the first image and the second image, as produced by the first imager and the second imager, respectively.

With reference to FIG. 5B, a first imager may produce the first image 164 of an organ of the body 242 in a display (not shown). The PRSs 160, 240 are represented by two marks 244, 246, respectively in the display, and the distance between the marks 244, 246 is designated by L₁.

With reference to FIG. 5C, a second imager may produce the second image 184 of the organ in the display. The PRSs 160, 240 are represented by two marks 248, 250, respectively in the display, and the distance between the marks 248, 250 is designated by L₂.

In the example set forth in FIGS. 5B and 5C, the scale of the first image 164 may be twice that of the second image 184 (i.e., L₁=2 (L₂)). Here, a scale factor of two is used merely for example. In order to provide the correct impression of the first image and the second image to a viewer (not shown), the first image and the second image may have to be displayed at substantially the same scale.

With reference to FIG. 5D, the processor may scale the first image 164 (not shown) by 50%, thereby producing another first image 252. The PRSs 160, 240 are represented by two marks 254, 256, respectively in the display, and the distance between marks 254, 256 is L₂ (i.e., the same as that between marks 248 and 250). Thus, the first image 252 and the second image 184 at substantially the same scale are more-appropriately sized for association.

Patient Table Motion Compensation.

During a medical procedure or between multiple medical procedures, the patient's body may move—both with regard to the operating table and locally. To properly associate data from one coordinate system to another coordinate system, motion compensation may be needed to account for this movement. In particular, the system may account for movement between the times when two images that are being associated were acquired.

The PRS provides a stable, positional reference of the patient's body so as to allow motion compensation for gross patient body movements. With respect to the coordinate system shown in FIG. 2 for frame of reference, movement of the patient along the operating table generally results in translational and rotational motion of the PRS in the X-Y plane. As described above, a PRS may be attached to the patient's manubrium sternum, a stable place on the chest, or some other location that is relatively stable. Alternatively, the PRS may be implemented by a multiplicity of physical sensors that are attached to different locations on the patient's body. Table motion by the patient may be addressed by using the P/O of the PRS as an anchor in reference coordinate systems I and II. Virtual anchors placed at anatomical landmarks can likewise aid in motion compensating patient movement along the operating table.

Cardiac Motion Compensation.

Images from the first and second time periods do not necessarily correspond to the same cardiac phase. This may be true even where the second imager acquires images of the body at P/Os similar or equal to the P/Os in which the first imager was arranged. This disparity in cardiac phases of associated images is undesirable because the heart takes on different shapes and sizes at different cardiac phases. One way to account for these differences in cardiac phase is to associate a second image with a first image that was acquired during the same, or at least a similar, cardiac phase. And even if the cardiac phase of the heart shown in the second image does not exactly match the cardiac phase of the heart shown in the first image, any residual error can be compensated for by a cardiac compensation function, as described more fully in U.S. Patent Publication No. 2011/0054308 to Cohen et al., entitled “METHOD AND SYSTEM FOR SUPERIMPOSING VIRTUAL ANATOMICAL LANDMARKS ON AN IMAGE,” which is hereby incorporated by reference in its entirety. Although a heart is used in this example, the concept of organ “phase matching” is not limited to cardiac phases where the inspected organ is a heart, but may instead apply to any body organ that experiences phases.

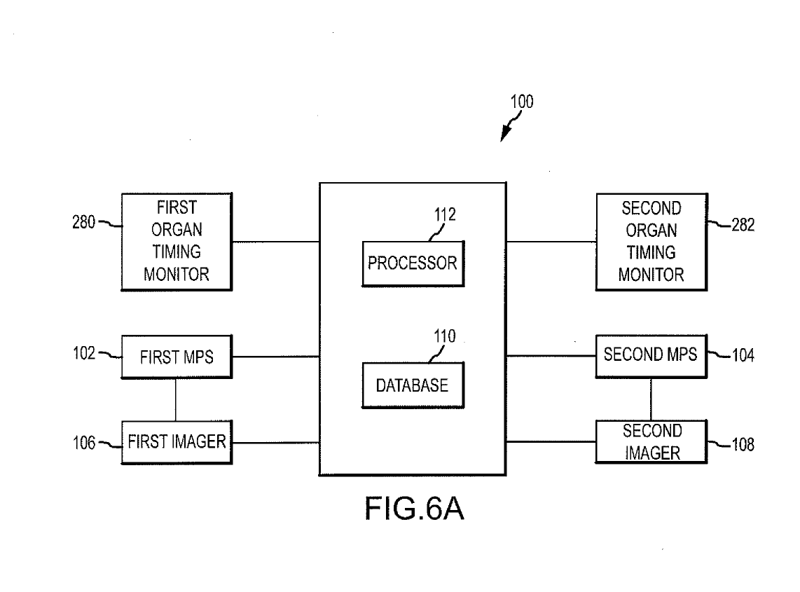

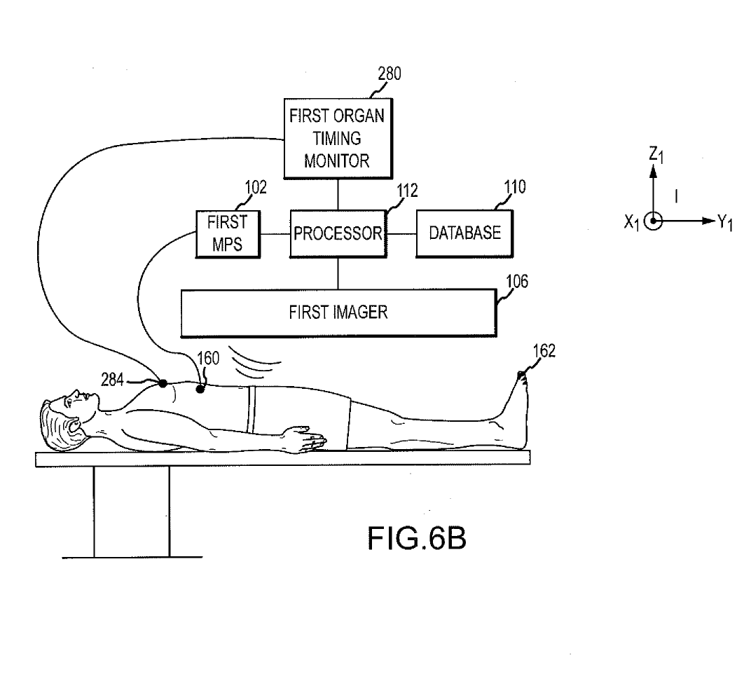

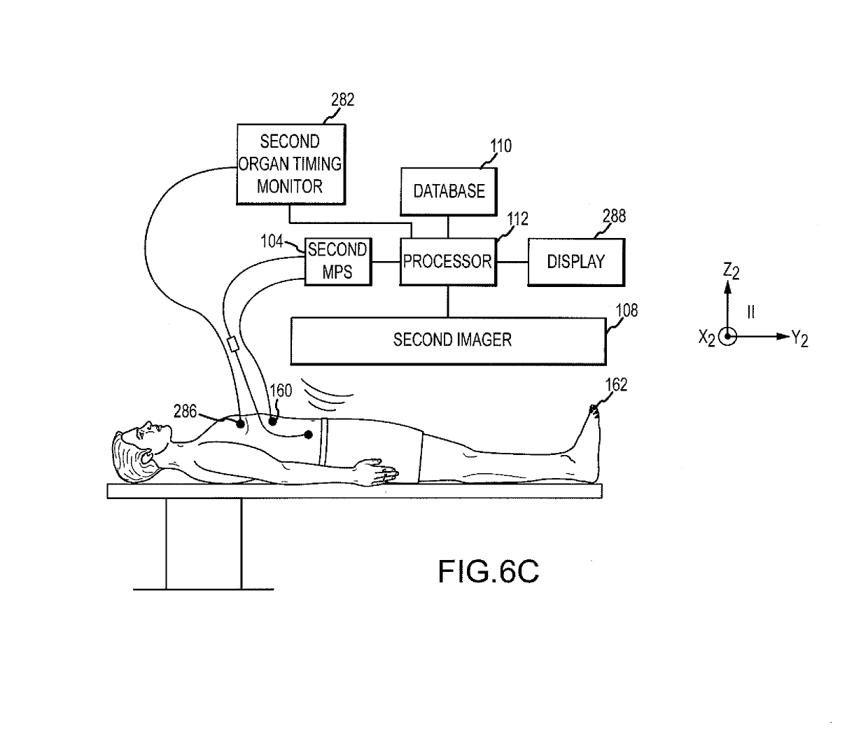

As shown in FIG. 6A, in addition to the devices described above, the system 100 may include a first organ timing monitor 280 and a second organ timing monitor 282 similar to those described in U.S. Patent Publication No. 2009/0182224 to Shmarak et al., entitled “METHOD AND APPARATUS FOR INVASIVE DEVICE TRACKING USING ORGAN TIMING SIGNAL GENERATED FROM MPS SENSORS” and U.S. Patent Publication No. 2011/0158488 to Cohen, entitled “COMPENSATION OF MOTION IN A MOVING ORGAN USING AN INTERNAL POSITION REFERENCE SENSOR,” which both are hereby incorporated by reference in their entireties. The first and second organ timing monitors 280, 282, which may be ECG monitors, for example, may monitor the electrical activity of the heart as a function of time. This electrical activity can reveal the current stage or phase of the heart within the cardiac cycle. Moreover, although the devices are shown in a particular arrangement in FIG. 6A, this arrangement is merely exemplary, and the present disclosure contemplates many different arrangements, such as that shown in FIG. 6B.

Each of the first and second organ timing monitors 280, 282 may be a device for monitoring the pulse rate of an inspected organ, such as the heart, the lungs, the eyelids, and the like. The organ timing monitors 280, 282 can continuously detect electrical timing signals of a heart organ, for example, through the use of a plurality of ECG electrodes (not shown) affixed to a patient's body. The timing signal generally corresponds to and is indicative of the particular phase of the organ (e.g., cardiac cycle) among other things.

With reference to FIG. 6B, which generally depicts apparatuses used in the first time period, the PRS 160 may be coupled with the first MPS 102, as described above. A first pulse sensor 284 or other organ sensor may be attached to an organ (not shown) of patient 162, such as the heart, and coupled with the first organ timing monitor 280. Although shown to be coupled via wires, the PRS 160, first pulse sensor 284, and other sensors may be coupled wirelessly to respective devices of the system 100.

The first imager 106 may acquire a plurality of 2D images from the body of the patient 162 and provide a signal respective of those 2D images to the processor 112. The first MPS 102 may also provide the P/O of the first imager 106 at the times when the 2D images are acquired. The first organ timing monitor 280 may determine the timing signal of the organ of the patient 162 according to a signal received from the first pulse sensor 284. The first organ timing monitor 280 may then provide a signal respective of the timing signal to the processor 112. The timing signal can be, for example, the QRS wave of the heart. The processor 112 may then associate each of the acquired 2D image signals with the P/O of the first imager 106, with the determined P/O of the PRS 160, and with the timing signal from the first pulse sensor 284. This set of data, for each acquired image, may then be stored in the database 110.

With reference to FIG. 6C, which generally depicts apparatuses used in the second time period, the processor 112 may be coupled with the second imager 108, with the second MPS 104, with the second organ timing monitor 282 or other organ sensor, and with the database 110. The second imager 108 may be coupled with the second MPS 104. The PRS 160 may be coupled with the second MPS 104. A second pulse sensor 286 may be coupled with the second organ timing monitor 282 and with the same organ that the first pulse sensor 284 was attached to in the first time period. As with the imagers and the MPS, the first organ timing sensor and monitor may in some embodiments be the same equipment as the second organ timing sensor and monitor—merely referred to as “first” and “second” for purposes of clarity and association with the first and second time periods. In other embodiments, however, the first organ timing sensor and monitor may in fact be different equipment than the second organ timing sensor and monitor.

In some embodiments, the processor 112 may direct the second imager 108 to P/Os in which the first imager 106 acquired images of the body. When the second imager acquires an image, the second MPS 104 may provide the processor 112 with signals respective of the determined P/O of the second imager, signals respective of the determined P/O of the PRS 160, and signals respective of the organ timing signal. The processor 112 may use this set of data to associate images from the first and second time periods for view on a display 288.

For example, the processor 112 may retrieve a first image from the image database 110 according to both the P/O of the second imager and the phase of the heart. After compensating for respiratory motion, as described below, and patient table motion, as described above, the processor 112 may associate the first image from reference coordinate system I with the second image from reference coordinate system II using an anchor or anchors, as described above. The resultant image may then be displayed on the display 288. Each resultant image may be stored in the database 110 along with all other measured data.

Respiratory Motion Compensation.

The procedures discussed above with regard to cardiac motion compensation are equally applicable to other body organs that experience cyclic, or relatively cyclic, motion. For example, an organ timing monitor may be used to monitor the phase of the lungs in first and second time periods, where association of data from the two periods is based on respiratory phase. The present disclosure, however, also contemplates motion compensating for both cardiac and respiratory functions. One technique for motion compensating for both cardiac and respiratory functions is described in U.S. Patent Publication No. 2009/0182224 to Shmarak et al., entitled “METHOD AND APPARATUS FOR INVASIVE DEVICE TRACKING USING ORGAN TIMING SIGNAL GENERATED FROM MPS SENSORS,” which is hereby incorporated by reference in its entirety.

As disclosed therein, one exemplary way in which such motion compensation is achieved is by continuously monitoring the positions of sensors as they are positioned within a patient's body, in the first and second time periods. Because cardiac motion and respiratory motion are cyclic in nature, periodic frequencies can be detected based on the position of a sensor when the sensor is maintained in a location for several cardiac and respiratory cycles. The specific frequencies relating to the cardiac motion exhibit different characteristics than the specific frequencies relating to the respiratory motion. The specific frequencies relating to the cardiac motion are identified from the detected periodic frequencies. Similarly, the specific frequencies relating to the respiratory motion are identified from the detected periodic frequencies. In effect, the system “learns” the motion at a given point within the patient's anatomy, and that motion can be broken down into two (or more) components: motion attributable to cardiac function and motion attributable to respiratory function. In turn, the P/O coordinates of a sensor at the moment when an image is acquired allow the system to determine the cardiac and respiratory phases of the patient's body at the time of that image.

It should be noted that this technique can also be used with virtual sensors, for respiratory and cardiac motion compensation, so long as the medical device “placing” the anchor is maintained at the position for several cardiac and respiratory cycles.

Yet if the first image from the first time period is selected to match the cardiac phase of the heart in the second image from the second time period, compensation for respiratory function is still needed. In this example, the P/O coordinates of sensors in both time periods may be neutralized such that motions due to respiratory functions are automatically filtered.

Exemplary Method of Operating the System.

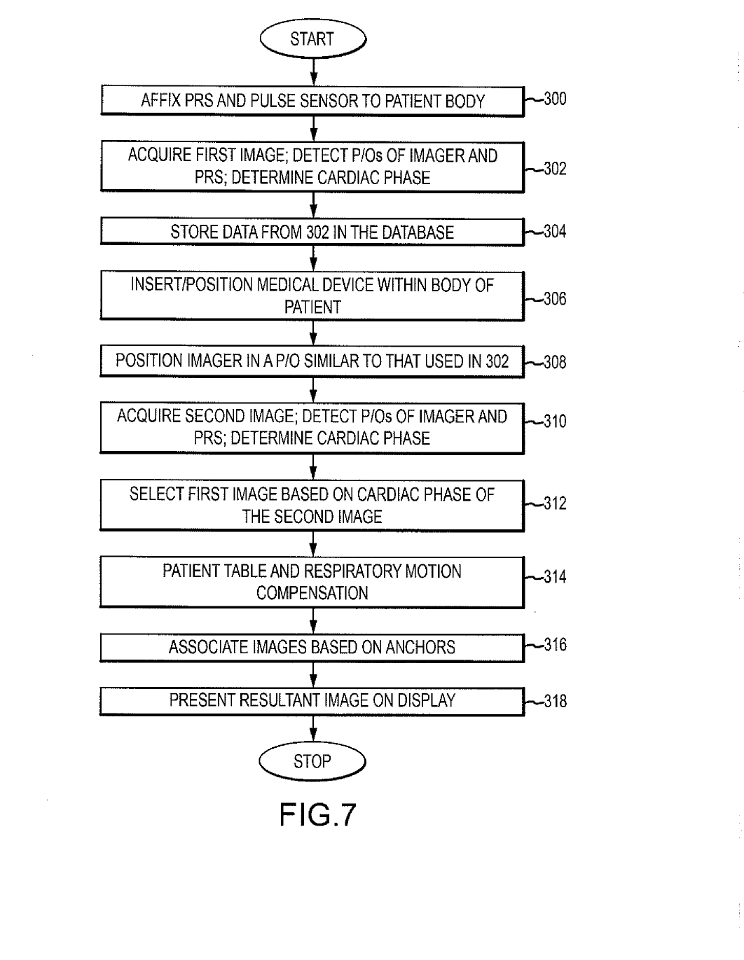

FIG. 7 depicts one of many possible methods of operating the system 100 in accordance with the disclosed embodiments. To begin, in step 300, a user of the system may affix at least one PRS and at least one pulse sensor to the body of a patient. The PRS may be placed in a relatively stable location along the patient's body such that it will remain substantially affixed to the same location during the first and second time periods. The pulse sensor, as described above, is helpful in determining the cardiac phase at the moment when an image of the patient body is acquired.

In step 302, the system may perform a number of actions either simultaneously or in quick succession. For one, an imager may acquire a first image or first images of the body of the patient. Also, as that image is acquired, an MPS may determine the positions of the imager and the PRS affixed to the body of the patient. The MPS may determine these positions based on signals supplied to the MPS from the PRS and/or a sensor affixed to the imager and/or identification of known features in the image. Further, at or around the same time the pulse sensor may detect a cardiac signal from which the system may determine the cardiac phase at the time when the first image was acquired. In step 304, these various pieces of datum may be stored as a record in a database for future retrieval.

In step 306, which may in some embodiments be part of the second time period, a medical device may be inserted within the body of the patient. The medical device may be radio-opaque such that the device is apparent on images that are subsequently acquired of the body of the patient. It should be understood that more than one medical device may be inserted into the body of the patient and that medical devices may already have been inserted within the body of the patient at this point, even though not described in this exemplary method.

In step 308, the imager may be arranged in a PLO that is substantially identical or similar to a P/O at which the imager acquired the first image. The P/Os may be defined with respect to the PRS affixed to the body of the patient.

Step 310, too, may involve a number of actions that occur simultaneously or in quick succession. First, the imager may acquire a second image of the body of the patient. Second, at or around the time when the second image is acquired, the pulse sensor may detect a cardiac signal from which the system 100 may determine the cardiac phase. Third, the MPS may determine the positions of the imager and the PRS affixed to the body of the patient.

In step 312, based on both the P/O of the imager and the cardiac phase corresponding to the second image, the system 100 may select first image for association with the second image. By associating images that correspond to the same cardiac phase, the system compensates for cardiac motion.

After the first image is selected, which may be part of a first set of images, the system in step 314 performs motion compensation as described herein to account for both patient movement along the operating table and motion caused by a patient's respiratory system. The P/O of the PRS in both the first and second time periods may be used to facilitate motion compensation for gross body movements, while the P/Os of internal sensors in the first and second time periods may be used to facilitate respiratory motion compensation.

In step 316, the first and second images may be associated using at least 3D anchor that is common to both images. The system may superimpose the images by making at least one of the images at least partially transparent or translucent and positioning the images over one another.

In step 318, the system may present the resultant image on the display. While the steps described here for displaying resultant images may repeatedly occur in real-time or near real-time as the medical device is navigated through the body of the patient, the user may opt to view the resultant images in slow motion or in a playback mode. Also, the system may repeat the steps of 300-304 many times during the first time period. Likewise, the system may repeat the steps of 306-318 many times during the second time period.

Use of the System in a CRT Implantation Procedure.

In one exemplary embodiment, the system may be used to enhance a cardiac resynchronization therapy (CRT) implantation procedure. In such a procedure, medical devices and a left ventricular (LV) lead are typically advanced through a patient's coronary sinus ostium. It is often desirable, therefore, to have representations of the medical devices in relation to the coronary sinus when maneuvering these medical devices through the body. One way to obtain a good image of the coronary sinus is to take a venogram of the anatomy, whether occlusive or non-occlusive. Taking an occlusive venogram may involve injecting contrast agent into the coronary sinus and trapping the contrast agent within the coronary sinus with the aid of a balloon catheter. Once a first set of images of the coronary sinus is acquired, the balloon catheter is then removed to create access for maneuvering the medical devices and the LV lead through the coronary sinus ostium. Once the medical devices are maneuvered within the body, a second set of images may be acquired. As opposed to highlighting anatomy with the injected agent, which begins to disperse after the balloon catheter is removed, the second set of images may reveal the position of the medical devices. By associating the first set of images highlighting the anatomy of the coronary sinus with the second set of images revealing the medical devices, the system presents a user with resultant images that show the medical devices in direct relation to the coronary sinus anatomy.

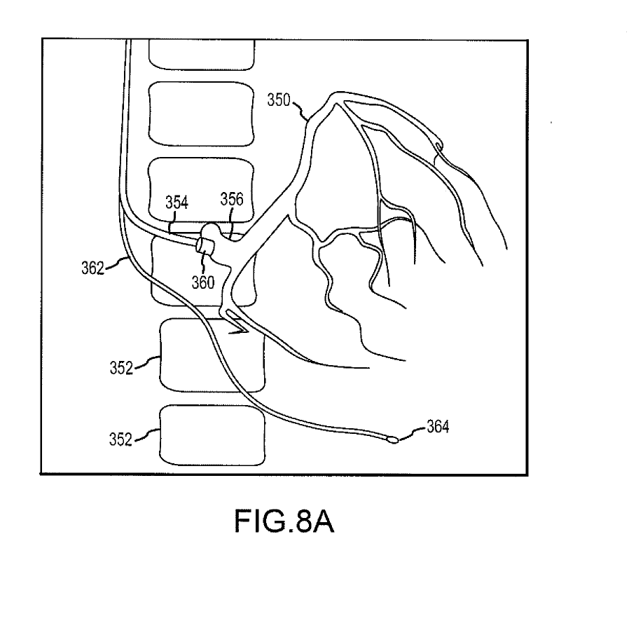

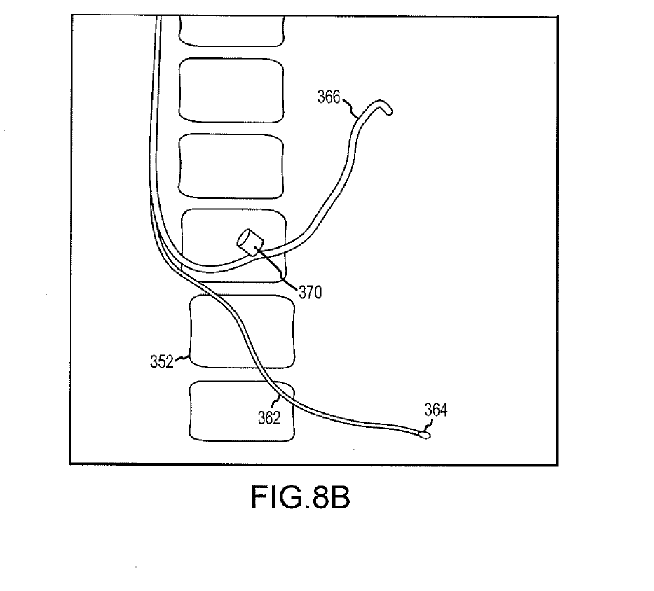

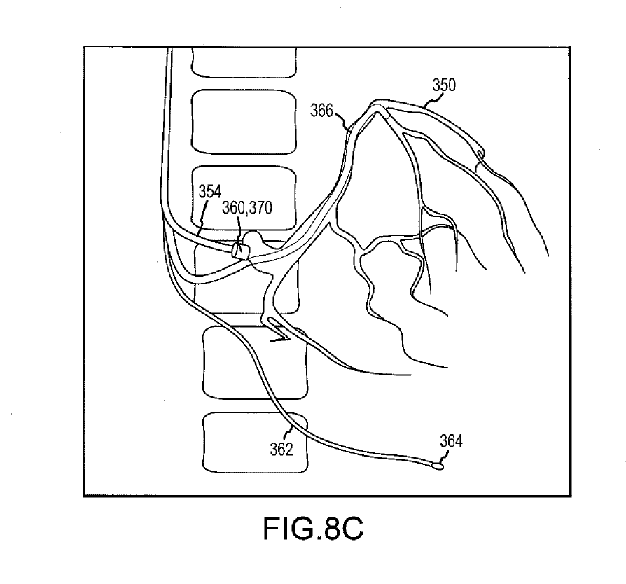

With respect to FIGS. 8A, 8B, and 8C, the enhanced CRT implantation procedure is described in more detail with reference to the components of the system described above. FIG. 8A shows a schematic illustration of an exemplary first image, here a venogram, which may have been acquired with a first imager (e.g., a C-arm fluoroscopic imaging device shown in FIG. 2) during a first time period. Contrast agent that has been injected into the coronary sinus highlights an anatomy 350 of the coronary sinus and its various branches, which appear in the forefront of vertebrae 352 of the patient body. A balloon catheter 354, moreover, has been maneuvered within a coronary sinus ostium 356 and occludes the coronary sinus so as to retain the contrast agent within the coronary sinus. In this configuration, a series of first images, like that shown by FIG. 8A, may have been acquired during this first time period when the coronary sinus was occluded. Ideally, the series of first images would represent a variety of cardiac phases from each of a variety of P/Os of the first imager, as measured by an MPS.

One virtual anchor and one physical anchor are also shown in the first image in FIG. 8A. The virtual anchor has been superimposed onto the first image. In the course of maneuvering the balloon catheter 354 to the coronary sinus ostium 356, an anatomical landmark 360 was identified at the coronary sinus ostium 356, within which the balloon catheter 354 may occlude the coronary sinus during the first time period. The 3D P/O of the anatomical landmark 360, as measured by an MPS, may serve as a virtual anchor so long as the landmark is again identified in the second time period. In addition, a reference catheter 362 having a distal sensor 364 has been maneuvered through a blood vessel near the coronary sinus. If left in place in the patient body during the first and second time periods, the 3D P/O of the distal sensor 364 may serve as a physical anchor for the association of images from the first and second time periods. Further, it should be understood that in the alternative, the reference catheter 362 may have placed the virtual anchor at the anatomical landmark 360.

Now referring to FIG. 8B, a second imager may capture a second image of the same region of the patient body in a second time period after the balloon catheter has been removed. In some embodiments the same imaging modality may be used during the second time period, while in others, a different imaging modality may be used. That said, the second image in FIG. 8B shows the reference catheter 362 and the distal sensor 364, as maintained in the patient body throughout the first and second time periods. A medical device 366 has been inserted through the coronary sinus ostium (not shown) and into the coronary sinus (not shown). Further, in the course of maneuvering the medical device 366 to the coronary sinus, an anatomical landmark 370 has been identified at the coronary sinus ostium, which may serve as a virtual anchor point.

Based on the cardiac phase detected when the second image is acquired, the system may select one of the images from the set of first images that has the same or a similar cardiac phase. For the sake of this example, the first image of FIG. 8A is presumed to correspond to a cardiac phase that matches that of the second image in FIG. 8B. After motion compensating the first and second images for respiratory and patient table motion in accordance with various embodiments described above, the system may associate the first and second images according to the physical and virtual anchors identified in FIGS. 8A-8B (e.g., anchors 360, 364, 370).

Once the first and second images are associated, as shown in FIG. 8C, the anatomy 350 of the coronary sinus from the first image is superimposed with the medical device 366 from the second image. This resultant image, which may be presented to a user on a display, offers a perspective of the anatomy 350 of the coronary sinus in relation to the medical device 366 that is used for delivering therapy. Further, this resultant image may be continuously updated to represent the current position of the medical device 366 within the body of the patient.

Another exemplary context in which the system may be used is in the field of coronary arterial interventions. Medical professionals often use cine-loops with contrast agent to visualize the target coronary sinus anatomy. Because contrast agents have an adverse impact on patients' renal functions, especially diabetic patients, it is desirable to minimize the use of contrast agents. Instead of using a contrast agent in the both the first and second time periods, a medical professional may use the system 100 to generate the cine-loop showing medical devices in relation to a patient's anatomy. In this instance, the contrast agent would only be needed during the first time period.

Although numerous embodiments of this disclosure have been described above with a certain degree of particularity, those skilled in the art could make numerous alterations to the disclosed embodiments without departing from the spirit or scope of this disclosure. For example, the disclosed techniques could be used to combine an image of an anatomy with a subsequent or live image of the anatomy. Or the disclosed techniques could be used to combine an image of an anatomy with an image showing anchors or an implanted device. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosed system and methods. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosed system and methods as defined in the appended claims.

What is claimed is:

1. A system for displaying an anatomy of a body of a patient, the system comprising:
an imager for acquiring a first image of the anatomy of the body in a first time period and for acquiring a second image in a second time period, the second image showing one or more of:
the anatomy of the body, and
a medical device positionable on or within the body;
a first anchor physically or virtually positionable on or within the body, the first anchor remaining substantially fixed on or within the body in the first and second time periods;
a database for storing the first image;
a medical positioning system (MPS) for determining three-dimensional (3D) positions of the first anchor in the first and second time periods;
a processor for associating the 3D positions of the first anchor with the first and second images and for associating the first image with the second image based on the 3D positions of the first anchor; and
a display for presenting a resultant image formed by the association of the first and second images.

2. The system of claim 1 wherein the resultant image is formed by superimposing the first and second images, with one or both of the first and second images being partially transparent.

3. The system of claim 1 wherein the first anchor is a sensor and is affixed to the medical device, the sensor being maintained within the body of the patient in the first and second time periods, wherein the MPS determines a 3D position of the sensor in each of the first and second time periods, wherein the processor associates the 3D position of the sensor from the first time period with the first image and associates the 3D position of the sensor from the second time period with the second image, wherein the processor uses the 3D positions of the sensor in the first and second time periods to associate the first and second images.

4. The system of claim 1 further comprising:
an organ sensor for sensing phases of a body organ in the first and second time periods; and
a first set of images acquired by the imager and stored in the database in the first time period;
wherein the first image is one of the first set of images, wherein the processor associates a sensed phase of the body organ with each of the first set of images and with the second image, wherein in the second time period the processor selects the first image from the first set of images based on a sensed phase of the body organ when the second image is acquired.

5. The system of claim 4 wherein the processor uses the 3D positions of the first and a second anchor in the first and second time periods to compensate for patient table movement between the first and second time periods and for movement caused by patient respiratory activities between the first and second time periods.

6. The system of claim 4 wherein the second image is one of a second set of images acquired during the second time period, wherein the processor updates the resultant image on the display in response to the acquisition of each of the second set of images.

7. The system of claim 6 wherein in the first time period the imager acquires the first set of images from two positions and orientations with respect to the first anchor, wherein in the first time period the first set of images are acquired during two phases of the body organ, wherein in the second time period the imager acquires the second set of images from one of the two positions and orientations.

8. The system of claim 7 wherein contrast agent is employed in an anatomical structure of the body in the first time period, wherein a representation of the contrast agent within the anatomical structure is visible in the first set of images, wherein the contrast agent is not present in the second set of images.

9. The system of claim 7 wherein the medical device is radio-opaque and is represented in the second set of images.

10. A method for displaying an anatomy of a body of a patient, the method comprising:
affixing an anchor physically or virtually on or within the body;
acquiring a first set of images of the anatomy of the body and storing the first set of images in a database;
acquiring a second image showing one or more of:
the anatomy of the body, and
a medical device disposed within the body;
determining a three-dimensional (3D) position of the anchor when each of the first set of images and the second image are acquired;
retrieving one of the first set of images from the database; and
associating the retrieved one of the first set of images with the second image based on the 3D positions of the anchor.

11. The method of claim 10 further comprising:
identifying a phase of a body organ as each of the first set of images and the second image are acquired; and
selecting the retrieved one of the first set of images based on the phase of the body organ at a time when the second image is acquired.

12. The method of claim 10 wherein associating the retrieved one of the first set of images with the second image further comprises:
rendering one or both of the retrieved one of the first set of images and the second image partially transparent; and
superimposing the retrieved one of the first set of images with the second image.

13. The method of claim 10 wherein the first set of images is acquired in a first time period and the second image is acquired in a second time period, wherein affixing the anchor on or within the body further comprises locating a virtual anchor within the body of the patient in each of the first and second time periods, with the virtual anchor point having a 3D position in each of the first and second time periods.

14. The method of claim 13 wherein associating the retrieved one of the first set of images with the second image further comprises using the 3D positions of the virtual anchor point from the first and second time periods for association.

15. The method of claim 10 wherein the first set of images is acquired in a first time period and the second image is acquired in a second time period, wherein affixing the anchor physically on or within the body further comprises:
positioning a sensor to a stable position on or within the body; and
maintaining the sensor in the stable position for the first and second time periods.

16. The method of claim 15 wherein acquiring the second image further comprises arranging an imager in a position and orientation that approximates a position and orientation in which one of the first set of images was acquired.

17. A system for displaying a medical device in relation to an anatomy of a body of a patient, the system comprising:
a first imager for acquiring a first set of images of the anatomy of the body in a first time period;
a second imager for acquiring a second set of images in a second time period, the second set of images showing the medical device disposed within the body of the patient;
a first organ sensor for sensing a phase of a body organ in the first time period as each of the first set of images is acquired;
a second organ sensor for sensing a phase of the body organ in the second time period as each of the second set of images is acquired;
a sensor affixed to the body of the patient, the sensor remaining substantially affixed to the body in the first and second time periods;
a first medical positioning system (MPS) for determining a three-dimensional (3D) position of the sensor in the first time period;
a second MPS for determining a 3D position of the sensor in the second time period;
a processor for associating the 3D positions of the sensor and the sensed phases of the body organ with the first and second set of images, the processor for associating one of the first set of images with one of the second set of images based on the 3D positions of the sensor and based on the sensed phases of the body organ; and
a display for presenting a resultant image formed by associating one of the first set of images and one of the second set of images.

18. The system of claim 17 wherein the resultant image is formed by superimposing and making transparent one of the first set of images and one of the second set of images.

19. The system of claim 18 wherein a virtual anchor is located within the body of the patient in the first and second time periods, wherein the processor uses 3D positions of the virtual anchor in the first and second time periods to associate the first set of images and the second set of images.

20. The system of claim 19 wherein the processor motion compensates the 3D positions of two virtual anchors to account for patient operating table motion and for motion caused by patient cardiac and respiratory activity.
